(19) United States
(12) Patent Application Publication
(10) Pub. No.: US 2015/0299107 A1
(43) Pub. Date: Oct. 22, 2015

(54) SELECTIVE EXTRACTION OF AN OMEGA-FUNCTIONALIZED ACID AFTER OXIDATIVE CLEAVAGE OF AN UNSATURATED FATTY ACID AND DERIVATIVES

(71) Applicant: ARKEMA FRANCE, Colombes (FR)

(72) Inventor: Markus Brandhorst, Lyon (FR)

(21) Appl. No.: 14/441,701

(22) PCT Filed: Nov. 7, 2013
(86) PCT No.: PCT/FR2013/052657

§ 371 Date: May 8, 2015

(30) Foreign Application Priority Data
Nov. 9, 2012 (FR) 1260643
Dec. 4, 2012 (FR) 1261611

(51) Int. Cl. C07C 253/34

(52) U.S. Cl. CPC C07C 253/34

(57) ABSTRACT

The invention relates to a process for the selective extraction of a reaction product, derived from an oxidative cleavage of an unsaturated fatty acid or of an ester derivative or of an unsaturated nitrile derivative, said reaction product being an ω-functionalized acid from among C₆ to C₁₅ diacids, acid esters and acid nitriles, using as selective extraction solvent a composition comprising a mixture of water and of C₁-C₄ carboxylic acid. The invention also relates to the use of said process and of the extraction solvent composition for the preparation of C₆ to C₁₅ amino acid, diacid or acid ester monomers for the preparation of polycondensation polymers, in particular polyamides.

The work which led to this invention received financial backing from the European Union within the framework of the 7th Programme Cadre (FP7/2007-2013) under the project number No. 241718 EUROBIOREF.

The present invention relates to a process for the selective extraction of an omega-functionalized (or ω-functionalized) C₁-C₁₅ aliphatic acid, bearing in the ω position an acid or ester or nitrile function, respectively, as a diacid, acid ester or acid nitrile (or cyano acid) from a reaction mixture obtained in particular after oxidative cleavage of an unsaturated fatty acid or including the ester derivative thereof (natural oil) or the nitrile derivative thereof, of renewable origin and of industrial quality, using as selective extraction solvent a specific mixture based on water and at least one C₁-C₄ and more particularly C₁-C₃ carboxylic acid, in specific proportions of water and acid. The invention also relates to the use of said specific mixture as a solvent for the selective extraction of said ω-functionalized acid. More particularly, the invention relates to the use of said extraction process in a process for preparing α,ω-diacid, ω-ester acid or ω-amino acid monomers that are suitable in particular for manufacturing polyamides. The natural oils and derivatives used for the oxidative cleavage are of limited purity. More particularly, the purity of said unsaturated fatty acid or the ester or nitrile derivative thereof does not exceed 95%, preferably does not exceed 90% and more preferentially ranges from 40% to 90% by weight. The oils of industrial quality used are known as RBD (Refined Bleached Deodorized) oils. These treatments have the object of eliminating the impurities present after the step of pressing the seeds, or even of the solvent extraction. Some of these impurities are light fatty acids, natural pigments present in the seeds, gums, phospholipids, etc.

The use of natural oils as a source of unsaturated fatty acids, other esters or nitriles thus poses a difficult problem insofar as they consist of a mixture of various saturated and unsaturated, possibly polyunsaturated, or even functionalized acids, which entails in the course of an oxidative treatment on fatty acid derivatives, especially when oxidative cleavage is used, the formation of a mixture of products:
desired ω-functionalized acids from among diacids, acid esters or ω-cyano carboxylic acids, especially of different chain lengths, while at the same time having the same functionalizations as a function of the position of the double bond(s) on the fatty chain,
intermediate products (epoxides, glycols),
   but also a significant proportion of byproducts and of unreacted or unreactive residual starting materials, which are saturated at the start, necessitating difficult or expensive separation treatments.

This remains valid whether the fatty acids concerned are hydroxylated or not. The separation of the ω-functionalized acid from the residual starting saturated derivatives, for example the separation of the ω-cyano acid from the saturated nitrile present at the start, is particularly complicated since the separation thereof by distillation is difficult on account of the similar boiling points. This problem posed by the presence in the starting oil of various unsaturated and corresponding saturated fatty acids leads after oxidative treatment (oxidative cleavage) to a multiplication of byproducts/co-products and of unreacted residual starting materials to be separated. It is all the more acutely perceived since, in the future, there will be ever-increasing reliance on GMO (genetically modified organisms) plants to improve productivity.

The problem arises more particularly for reaction byproducts which include unreacted residual starting materials, after oxidative cleavage reaction, and which have corresponding molecular masses and/or boiling points, which are at least as high as the corresponding characteristics of the ω-functional acid reaction product to be separated/purified. It is clear that the problem arises for all the heavier byproducts or unreacted or unreactive residual products and those that are very close to said ω-functional acid product, such that separation by distillation remains difficult, on the one hand due to the high boiling points of the products to be separated, which entails a risk of thermal degradation at high temperature (boiling range) and, on the other hand, due to the fact that several byproducts have very similar boiling points and it is therefore difficult to separate them via this route. More particularly, the separation of the products obtained by oxidative cleavage of a fatty acid or of a fatty nitrlle is a key step. Fatty acids are noted CX:Y, with X corresponding to the chain length and Y corresponding to the number of unsaturations. Furthermore, the double bond may be in cis or trans conformation. The names delta-Z or omega-W may be added, Z then denoting the position of the double bond in the chain counting from the acid group and W denoting the position from the other end of the chain. The presence of saturated fatty chains (C14:0, 16:0, 18:0 and 20:0 according to the CX:Y nomenclature) in the starting material leads to the formation of a mixture after oxidative cleavage that will be difficult to separate by distillation. The boiling points of acid nitriles and saturated nitriles in the case of cleavage of nitrile or of diacids and of saturated acids in the case of cleavage of acid are very similar, although being of different chain length. Thus, for example, the diacid containing 9 carbon atoms has a boiling point close to palmitic acid C16:0 and stearic acid. Selective extraction of the acid nitrile or diacid relative to the saturated fatty nitrile or saturated fatty acid using a carboxylic acid/water mixture has been explored.

Consequently, there is a need for a non-thermal separation method, avoiding thermal degradation of the product, which can lead to additional byproducts to be separated and ensure the thermal and chemical stability of said ω-functional acid product (or corresponding ester/nitrile) to be separated, having a high separation yield and final purification yield and an implementation that is also simple and practical and free of complicated and expensive steps.

GB 1 177 154 describes, in example 15, the use of an acetic acid/water mixture (88.9/11.1) as solvent for the hydrogenation of 11-cyanoundecanoic acid to obtain 12-aminododecanoic acid. Similarly, said document describes the use of acetic acid as a suitable solvent for the hydrogenation of this compound. The problem described in said document is linked to the hydrogenation of 11-cyanoundecanoic acid and does not in any way concern the selective separation of this acid from a multitude of formed byproducts (saturated and unsaturated) following an oxidative cleavage of an unsaturated fatty acid nitrile, as is the case in the present invention. Furthermore, an acetic acid/water mixture (88.9/11.1 by volume) does not allow separation of the targeted cyano acid from a saturated nitrile.

Perkins et al. describe in JACS, 1975, 52, 473, from the final paragraph of page 473 to the first paragraphs of page 474, the preparation of methyl 8-cyanooctanoate by oxidative cleavage of oleonitrile via ozonolysis using as solvent for this ozonolysis acetic acid in the presence of water (implicit mixture corresponding to 90/10). After removal of the acetic acid by distillation, the crude mixture obtained is esterified with methanol using dichloromethane as esterification solvent, which is removed by distillation, after washing the medium with aqueous bicarbonate. Next, the crude mixture of esters obtained is distilled under vacuum. No disclosure or suggestion of use of a specific mixture of acetic acid and water for selectively extracting 8-cyanooctanoic acid is described in said document.

U.S. Pat. No. 4,165,328 describes a process for the separation-recovery of 11-cyanoundecanoic acid with a degree of purity of between 75% and 95% by weight starting with a mixture containing cyclohexanone and ε-caprolactam, this mixture being derived from the pyrolysis between 300 and 1000° C. of 1,1′-peroxydicyciohexylamine. The mixture is first treated with a mixture of aqueous ammonia solution and of a solvent from among benzene, xylene and toluene, with passage of the cyclohexanone into the organic phase and of the ammonium salt of 11-cyanoundecanoic acid into the aqueous phase. The 11-cyanoundecanoic acid is recovered after acidification of this phase at 40-100° C. first in crude and molten form and then washed with hot water to remove the ε-caprolactam. This process remains specific to the production of this acid nitrile from a specific starting material and specific conditions, which do not apply to the problem posed in the case of the oxidative cleavage of an unsaturated fatty acid or of an ester or nitrile derivative of this fatty acid. The process generates as byproduct 1 mole of salt per mole of cyano acid, which salt it is necessary either to remove/retreat as waste, or to retreat/upgrade as usable material. This process thus requires additional steps for treating this byproduct and, as such, it is therefore neither practical nor flexible.

GB 1 049 229 describes the preparation of an ω-cyano aliphatic carboxylic acid from the ammoniation of the corresponding α,ω-diacid, for example the production (according to example 1) of 9-cyanononanoic acid from 1,10-decanedioic diacid treated with urea, with, as byproducts to be separated out, the corresponding dinitrile and the ω-cyanocarboxamide corresponding to the starting diacid. Dissolution in a dilute aqueous ammonia solution and extraction with a water-immiscible solvent such as ether makes it possible to extract the neutral products such as the dinitrile and the cyano-carboxamide compound, from the cyano-carboxylates and the dicarboxylates. Next, the ammoniacal phase is distilled and extracted with chloroform, in which the 9-cyanononanoic acid is found, and the residual 1,10-decanedioic diacid is recovered in the residual ammoniacal phase after distillation. Once again, this process bears no relation to an oxidative cleavage reaction and the byproducts associated with this reaction or with the process of the present invention.

U.S. Pat. No. 3,994,942 describes a process for recrystallizing 11-cyanoundecanoic acid from a solvent which is an acetic acid/water or propionic acid/water mixture, but does not teach or describe this solvent as a selective liquid-liquid extraction solvent.

U.S. Pat. No. 2,468,436 describes a process for preparing an ω-cyanocarboxylic acid such as 8-cyanooctanoic acid prepared by oxidation of oleonitrile with a solution of chromic acid and of concentrated sulfuric acid, with the reaction mixture subjected to an extraction with petroleum ether which separates the pelargonic acid as acid byproduct from the unreacted nitriles, including the saturated nitriles present at the start in the oleonitrile used. The residue that is insoluble in petroleum ether contains 8-cyanooctanoic acid, which may be purified by conversion into the barium salt and hot dissolution in an excess of barium chloride with filtration of the impurities before conversion into the add form with sulfuric acid. Although this process uses the oxidation of oleonitrile, the separation and purification method appears complex and involves several different steps with use of materials that are impractical to use, and this process bears no relation whatsoever to the process of the present invention. Once again, this process involves the formation of a salt, as byproduct, with the same drawbacks as those mentioned above for a similar process, i.e. being impractical and inflexible.

The article entitled “Ozonolysis of unsaturated fatty acids I. ozonolysis of oleic acid” by Ackman et al. in Can. J. Chem., 39 (1961) pp. 1956-1962 describes the ozonolysis of pure oleic acid in methanol used as reactive solvent, with decomposition in formic acid with hydrogen peroxide of the ozonide formed. This leads to dicarboxylic acids obtained by oxidative cleavage as main products and a minimum amount of acid side products. Other ozonolysis solvents for the pure oleic acid are also described, such as acetic acid or acetone, like the oxidative cleavage of pure azelaic acid in formic acid as reaction solvent. However, said document of academic interest concerns only the oxidative cleavage of pure unsaturated fatty acids with absence at the start of equivalent saturated fatty acids and other starting materials (saturated and unsaturated) as is the case in an unsaturated fatty acid of industrial quality and of limited purity, as explained at the start. In such a case, the composition of the cleavage reaction products and of the starting saturated products is much more complex than that described in the cited document with a significantly large proportion of reaction byproducts and of unreactive residual products to make their removal much more difficult and complex than that described in said document.

The solution proposed by the present invention overcomes the drawbacks of the methods known in the prior art, with a specific and selective process for extracting said ω-functionalized acid from the reaction mixture, in particular derived from an oxidative cleavage of an unsaturated fatty acid or ester thereof (including an oil form) or unsaturated fatty acid nitrile thereof, said process being based on the use of a specific extraction solvent composition.

The main object of the invention first concerns said specific process for extracting an ω-functionalized acid from a reaction mixture derived from an oxidative cleavage, followed by a process for manufacturing said acid or derivative thereof (in the case of an amino acid from an acid nitrile), comprising the use of said selective separation process and finally the use of the specific composition of the selective extraction solvent in such a process for extracting or manufacturing said acid, in particular for preparing α,ω-functionalized amino acid, diacid or acid ester acidic monomers.

Thus, the first subject of the invention concerns a process for the selective separation of a reaction product from the reaction mixture and relative to reaction byproducts and unreacted or unreactive residual starting materials, in which:
a) said reaction product is at least one ω-functionalized acid, in particular selected from diacids, acid esters and acid nitriles and comprising from 6 to 15 carbon atoms,
b) said product is derived from a reaction for the oxidative cleavage of at least one unsaturated fatty acid, including a hydroxylated acid or ester derivative thereof, including a monoester or multiester of a polyol, including glycerol or the nitrile derivative thereof,
c) said process comprises at least one step and, more particularly, several successive steps of selective extraction of said ω-functional acid product with a selective extraction solvent, which is a composition comprising a mixture of water and of at least one carboxylic acid containing 1 to 4 carbon atoms (or C₁ to C₄) or mixtures thereof, preferably 1 to 3 carbon atoms or mixtures thereof, and more preferentially said acid being acetic acid or formic acid or a mixture thereof, more particularly the mixture of formic acid and acetic acid and in a water/acid ratio such that, at the extraction temperature, the mixture of said reaction mixture with said extraction solvent is two-phase, said reaction product, the ω-functional acid, being selectively concentrated in the aqueous extraction phase and said reaction byproducts and unreacted or unreactive residual starting materials being concentrated in the organic phase at each extraction step,
d) said process comprises the treatment of said aqueous extraction phase, optionally cumulated over several extractions with removal by evaporation of said extraction solvent, water and acid and recovery of said product with the dry extract thus obtained (recovery of said product in the form of the dry extract obtained), optionally, said product possibly being recovered by liquid-liquid back-extraction using said aqueous phase with a solvent for said product which is immiscible with said aqueous phase.

As solvent that is suitable for said back-extraction, i.e. counter-current extraction, an organic solvent that is immiscible with the water/carboxylic acid mixture as described according to the invention and which has particular affinity, i.e. is a good solvent relative to said product to be extracted, may be chosen. This optional variant has an energetic advantage, i.e. with less energy consumed for this recovery variant, relative to the removal of the water and of said carboxylic acid by evaporation in said aqueous extraction phase.

The composition of said extraction solvent may vary and may be adjusted as a function of the extraction temperature, for a given water/carboxylic acid mixture. The composition may also vary as a function of the carboxylic acid used and also as a function of the desired yield/selectivity compromise. More particularly, the shorter the carboxylic acid, ranging from C₄ to C₁, the better the extraction selectivity and the longer the carboxylic acid, ranging from C₁ to C₄, the better the yield or the degree of extraction.

In the process according to the invention, said extraction may take place at a temperature ranging from the melting point to the boiling point of said water/carboxylic acid mixture, preferably from 15 to 70° C. and more preferentially from 15 to 50° C. Increasing the temperature has a tendency to reduce the selectivity and consequently, for a given water/acid ratio, the lowest temperature promotes the extraction selectivity. At the chosen temperature, the organic phase to be extracted (reaction mixture) remains liquid. Various extraction stages, in cascade and continuous, may be envisaged with extraction solvent mixtures and temperatures that may vary (gradient of extraction solvent composition and/or of temperature, from one stage to another with recycling of the residual streams into other stages suited to their composition, these gradients being suited to the starting compositions to be extracted (nature and proportion of products to be extracted in these compositions)).

More particularly, the temperature of said extraction step c) and said water/acid ratio are chosen such that, during this extraction step, the mixture of the water/acid composition with the reaction mixture (organic reaction phase) comprising said product to be separated out is two-phase with an aqueous phase (water-acid) selectively and predominantly containing said product to be separated out and a nonaqueous phase containing the rest of the organic phase.

Thus, the water/carboxylic acid weight ratio in said extraction solvent of said extraction step c) may vary as a function also of the extraction temperature used and of the acid (nature of the acid), from 32/68 to 95/5 and preferably from 38/62 to 85/15.

Preferably, said extraction according to the process of the invention takes place at room temperature, which means 20±5° C.

According to a particular option, said extraction step c) comprises several successive washes-extractions of said nonaqueous (organic) phase with said water/acid composition, with recovery and mixing of all the aqueous phases thus obtained before said treatment d) to recover said ω-functional acid product. The extractions may be performed with the same weight proportions between the extraction solvent and the reaction mixture to be extracted, but, preferably, the weight ratio between the extraction solvent and the weight of the reaction mixture to be extracted varies within a range from 0.5/1 to 100/1 and more preferentially from 0.5/1 to 50/1. More particularly, this ratio is reduced with the number of successive extractions, so as to improve the extraction selectivity of the targeted product.

More particularly, in the case of the selective extraction according to the process of the invention of an ω-cyano acid, from a reaction mixture derived from an oxidative cleavage, for example of 8-cyanooctanoic acid derived from the oxidative cleavage of oleonitrile comprising other saturated nitriles, the initial ratio between the acid nitrile to be extracted and the other saturated nitriles may pass from about 1 at the start to more than 300 after said selective extraction. This clearly demonstrates the advantage of the process of the present invention.

According to a preferred particular case, said selective separation process forms an integral part of the process for manufacturing said ω-functionalized acid.

The invention also covers a process for manufacturing an re-functionalized acid, in particular an α,ω-diacid, an ω-acid ester, an ω-acid nitrile or a derivative of these products and in particular an ω-amino acid as derivative of an ω-acid nitrile, which comprises the use of a selective separation process, as defined above according to the invention, and which manufacturing process comprises a step, prior to this use, of a reaction for the oxidative cleavage of an unsaturated starting material, from which reaction are derived said ω-functionalized product and said unreacted or unreactive residual starting materials and byproducts, said starting material subjected to said oxidative cleavage being chosen from at least one unsaturated fatty acid, preferably containing at least 10 carbon atoms and more preferentially at least 16 carbon atoms and/or an ester derived from said unsaturated fatty acid and/or a nitrile derived from said unsaturated fatty acid of formula (I) below:

[R1-CH═CH—[(CH₂)_{q}—CH═CH]ₘ—(CH₂)ᵣ]ₚ—X (I)

in which
p: is an integer equal to 1, 2 or 3 and
for p=1, X is chosen from: —CO₂H (fatty monoacid) or —CN (nitrile) or —CO₂R′ (monoester) with R′ being a C₁ to C₁₁ alkyl, the alkyl possibly being linear or branched when this is possible, i.e. from C₄ upwards,
for p=2, X is a diester radical ═(CO₂)₂—Y, with Y being a diol residue or glycerol residue bearing an OH function,
for p=3, X is a triester ═(CO₂)₃—Z, with Z being a glycerol residue (trivalent radical without OH) (in the case of a fatty acid oil)
   R1: is an H or an alkyl radical of 1 to 11 carbon atoms, where appropriate comprising an OH function,
   q: is equal to 0 or 1,
   m: is equal to 0, 1 or 2,
   r: is an integer ranging from 4 to 15,
   with the C═C unsaturations of said formula possibly being of cis or trans conformation.

More particularly, according to this manufacturing process, said unsaturated starting material of formula (I) is chosen from a fatty acid or a fatty acid ester, preferably from oleic acid, oleic oil or an oleic acid monoester and more preferentially oleic acid.

According to another variant, said unsaturated starting material (I) is a nitrile, preferably oleonitrile or octadec-11-enoic nitrile and octadec-12-enoic nitrile or mixtures thereof, synthesized from ricinoleic acid after hydrogenation followed by dehydration. Said nitrile according to (I) is in particular the product of ammoniation (reaction with ammonia) of the corresponding fatty acid or of an ester of said fatty acid and in particular of the corresponding oil. Said oxidative cleavage reaction step is preferably performed using as oxidative cleavage agent hydrogen peroxide, oxygen and/or ozone and in particular hydrogen peroxide.

According to a particular and preferred case according to this manufacturing process, said unsaturated starting material (I) is oleonitrile and said oxidative cleavage reaction product is 8-cyanooctanoic acid.

According to another particular and preferred case according to this manufacturing process, said unsaturated starting material (I) is gondoic acid (eicos-11-enoic acid) nitrile or vaccenic acid (octadec-11-enoic acid) nitrile and the oxidative cleavage reaction product thus obtained is 10-cyanodecanoic acid.

Said reaction byproducts and/or unreacted or unreactive heavier residual starting materials may in particular comprise:
saturated fatty acids, esters or nitriles of the same rank as said unsaturated starting material,
the residual unsaturated starting material of the diol or epoxy derivatives of said unsaturated starting material, formed by oxidative modification other than oxidative cleavage of the ethylenic unsaturation of said unsaturated starting material,
higher saturated and/or unsaturated fatty acids.

Said process for manufacturing an ω-functionalized acid preferably relates to the manufacture of an α,ω-diacid, an ω-acid ester or an r-amino acid as derivative of an ω-cyano acid, comprising from 6 to 15 carbon atoms, said manufacturing process using a selective separation process, as defined above according to the invention for the selective separation of said α,ω-diacid, ω-acid ester and ω-cyano acid. Such a more preferred process relates to the preparation of an ω-amino acid comprising from 6 to 15 carbon atoms obtained from the r-cyano acid (or acid nitrile) precursor thereof, said precursor being obtained via a process as defined according to the invention above, in particular by oxidative cleavage of a nitrile derivative of an unsaturated fatty acid, preferably comprising at least 10 carbon atoms and more preferentially at least 16 carbon atoms, as defined above and according to formula (I) mentioned above, with said manufacturing process comprising an additional step of hydrogenation of said ω-cyano acid precursor to obtain said amino acid. More preferentially, said (ω-cyano acid is 8-cyanooctanoic acid and said ω-amino acid derivative is 9-aminononanolc acid or said cyano acid is 10-cyanodecanoic acid and said amino acid derivative is 11-aminoundecanoic acid or said cyano acid is 11-cyanoundecanoic acid and said amino acid is 12-aminododecanoic acid or said cyano acid is 12-cyanododecanoic acid and said amino acid is 13-aminotridecanoic acid. More particularly, the 10-cyanodecanoic acid and 11-undecanoic acid may be obtained as a C₁₁/C₁₂ mixture after oxidative cleavage of the hydroxylated fatty acid 12-hydroxystearic acid or 12HSA (obtained by hydrogenation of ridnoleic acid) subjected beforehand (12HSA) to a dehydration with formation of a C₁₁ unsaturation (between C₁₁ and C₁₂) or a C₁₂ unsaturation (between C₁₂ and C₁₃).

The present invention also covers the use of a composition comprising a water/carboxylic acid mixture chosen from at least one C₁ to C₄ organic acid or mixtures thereof, preferably formic acid and/or acetic acid and/or propionic acid or mixtures thereof and more preferentially formic or acetic or propionic acid or mixtures thereof, even more preferentially acetic acid or formic acid or mixtures thereof, in particular the mixture of formic acid and acetic acid, as solvent for the selective extraction and separation-purification of at least one reaction product chosen from an α-ω-diacid, ω-acid ester, ω-cyano acid (or acid nitrile), comprising from 6 to 15 carbon atoms, starting with a reaction mixture comprising reaction byproducts and/or unreacted or unreactive heavier residual starting materials, the reaction mixture derived from a reaction for the oxidative cleavage of an ethylenically unsaturated starting material chosen from at least one unsaturated fatty acid including a hydroxylated fatty acid or an ester derivative including a monoester or multiester of a polyol including glycerol or a nitrile derivative of said unsaturated fatty acid, preferably containing at least 10 carbon atoms and more preferentially at least 16 carbon atoms, the water/carboxylic acid ratio in said extraction solvent being adjusted such that, at the extraction temperature, the mixture of said solvent with said reaction mixture is two-phase at said temperature. More particularly, in this use, the water/carboxylic acid ratio may range, as a function of the extraction temperature and of the carboxylic acid used, from 32/68 to 95/5 and preferably from 38/62 to 85/15. This use is of particular interest when said reaction product is 8-cyanooctanoic acid obtained from the oxidative cleavage of oleonitrile or when said reaction production is azelaic acid (nonanedioic acid) obtained from the oxidative cleavage of oleic acid or said product is the monoester of azelaic acid and obtained from an oleic acid ester or when said product is 10-cyanodecanolc acid and obtained from the oxidative cleavage of gondoic acid nitrile and/or from vaccenic acid nitrile.

Finally, the use of a process for the selective extraction or separation as defined according to the invention described above for the preparation of ω-amino acid, α,ω-diacid or ω-acid ester monomers as monomers for the manufacture of polymers for (or by) polycondensation, such as polyamides or polyesters, in particular polyamides, also forms part of the present invention. This process is of particular interest in the preparation of diacid or amino acid monomers for polyamides and more particularly in the preparation of 9-aminononanoic acid for the preparation of polyamide PA 9 or in the preparation of 11-aminoundecanoic acid for the preparation of polyamide PA 11 or in the preparation of 12-aminododecanoic acid for the preparation of polyamide PA 12 or in the preparation of 13-aminotridecanoic acid for the preparation of polyamide PA 13. Thus, the present invention also relates to the use of the process as defined above according to the invention in the preparation of 9-aminononanoic acid for the preparation of polyamide PA 9 or in the preparation of 11-aminoundecanoic acid for the preparation of polyamide PA 11 or of 12-aminododecanoic acid for the preparation of polyamide PA 12 or of 13-aminotridecanoic acid for the preparation of polyamide PA 13. Consequently, said selective separation process according to the invention may form part of a process for preparing amino acid monomers in a process for preparing polyamides, in particular PA 9, PA 11, PA 12 and PA 13. 13-Aminotridecanoic acid may be obtained from 12-cyanododecanoic acid, the latter as a product of the oxidative cleavage of erucic (doeicos-13-enoic) nitrile and selective extraction according to the process of the present invention.

The examples that follow are given as illustrations of the invention and of Its performance qualities and do not in any way limit the scope of the claims.

### EXAMPLES

### Starting Materials Used (See Tables 1a and 1b Below)

TABLE 1a
carboxylic acids in extraction solvent

| Starting material | | | Origin |
|---|---|---|---|
| (name) | Function vs invention | Purity | (supplier) |
| Formic acid (FA) | Acid in extraction | 98% | Prolabo |
| | solvent | | |
| Acetic acid (AA) | Acid in extraction | 100% | Prolabo |
| | solvent | | |
| Propionic acid (PA) | Acid in extraction | 99% | Aldrich |
| | solvent | | |

The water used in the mixture for the extraction solvent is demineralized water.

TABLE 1b
reaction mixtures to be extracted

| | | | Reaction vs | Reaction |
|---|---|---|---|---|
| | Product to | Starting | starting | mixture to be |
| Examples | be extracted | material | material | extracted |
| 1 | 8-Cyanooctanoic | Oleonitrile | Oxidative | R1 |
| | acid or 8COA | | cleavage | |
| | (ω-cyano acid) | | | |
| 2 | Azelaic acid or | Oleic acid | Oxidative | R2 |
| | AZ (diacid) | | cleavage | |

### Example 1

### Extraction of 8-Cyanooctanoic Acid from a Reaction Mixture R1 Derived from the Oxidative Cleavage of Oleonitrile

The reaction mixture R1 used is a solution having a composition as presented in table 2 below (according to analysis by gas chromatography).

TABLE 2
composition of the reaction mixture R1

| Reaction mixture R1 component | weight % | mmol/g |
|---|---|---|
| Nonanal | 0.7 | 0.049 |
| Heptanoic acid (A7) | 1.9 | 0.146 |
| Octanoic acid (A8) | 1.2 | 0.080 |
| Nonanoic acid (A9) | 12.4 | 0.785 |
| Decanoic acid (A10) | 0.5 | 0.028 |
| 7-Cyanoheptanoic acid (7CHA) | 1.6 | 0.105 |
| 8-Cyanooctanoic acid (8COA) | 19.8 | 1.169 |
| 9-Cyanononanoic acid (9CNA) | 1.0 | 0.053 |
| Myristic nitrile (14:0) | 1.7 | 0.080 |
| 10-Cyanodecanoic acid (10CDA) | 1.2 | 0.062 |
| Palmitic nitrile (16:0) | 4.3 | 0.182 |
| Stearic nitrile (18:0) | 1.8 | 0.068 |
| Arachidic nitrile (20:0) | 0.5 | 0.016 |

This mixture is obtained from the oxidative cleavage of an oleonitrile (purity of 80%, from Arkema). The cleavage was performed in a batch reactor at 80° C. for 24 hours using tungstic acid as catalyst and aqueous hydrogen peroxide solution (at 70% by weight of H₂O₂) at 63% by weight of pure H₂O₂ relative to the oleonitrile (pure). A stream of air passed through the reaction mixture.

The mole ratio of starting 8-cyanooctanoic acid (8COA) to Σ(sum) of the saturated nitriles (myristic nitrile+palmitic nitrile+stearic nitrile+arachidic nitrile) is (8COA)/I saturated nitriles=3.4. The reaction mixture used corresponds to that obtained after washing with water.

### Extraction Procedure

For the extraction, about 2 g of solution R1 were placed in contact in a separating funnel with about 20 g of a solution of a preformed mixture of carboxylic acid/water at the indicated temperature. The two solutions were mixed vigorously for about 1-2 minutes. After more than 30 minutes, the aqueous phase was separated out, the carboxylic acid and the water were evaporated under vacuum <1 mbar, 40° C. and the remaining product was analyzed by gas chromatography. For the tests with two carboxylic adds, the acids were premixed (50/50 weight ratio) and then mixed with water to form the composition of the extraction solvent.

Determination of the Concentration of Carboxylic Acid to be Mixed with Water

The oxidative cleavage solution is soluble in the carboxylic acid. To determine the amount of water necessary to obtain two separate phases, the water was gradually added. After shaking in a separating funnel, the funnel was left to stand for at least 5 minutes. The presence of a meniscus after 5 minutes was used to determine the minimum water/carboxylic acid ratio as presented in table 3 below.

TABLE 3
determination of the acid/water ratio

| | Amount of | | | |
|---|---|---|---|---|
| | carboxylic | Amount | Temper- | Formation |
| | acid | of water | ature | of a |
| Carboxylic acid | (weight %) | (weight %) | (° C.) | meniscus |
| Formic acid (FA) | 53 | 47 | 20 | No |
| Formic acid (FA) | 49 | 51 | 20 | Yes |
| Acetic acid (AA) | 64 | 36 | 20 | No |
| Acetic acid (AA) | 61 | 39 | 20 | Yes |
| Acetic acid (AA) | 68 | 32 | 60 | No |
| Acetic acid (AA) | 66 | 34 | 60 | Yes |
| Propionic acid (PA) | 55 | 45 | 20 | No |
| Propionic acid (PA) | 53 | 47 | 20 | Yes |
| 50/50 by weight | 71 | 29 | 20 | No |
| acetic acid/formic | | | | |
| acid mixture | | | | |
| (50/50 AA/FA) | | | | |
| 50/50 by weight | 68 | 32 | 20 | Yes |
| acetic acid/formic | | | | |
| acid mixture | | | | |
| (50/50 AA/FA) | | | | |

### Extraction Selectivity (See Table 4 Below)

TABLE 4
8COA/saturated nitrites mole ratio and other yield and selectivity
characteristics as a function of certain extraction parameters

| | | | | Weight of | | | Dry extract | 8COA |
|---|---|---|---|---|---|---|---|---|
| | | | Weight of | reaction | | 8COA/ | (100% material, | extracted |
| Test | | Acid/water | acid/water | mixture R1 | Temp. | Σnitrileₛₐₜ | without solvent) | vs starting |
| No. | Acid | (by weight) | (g) | (g) | (° C.) | (in mol/mol) | (mg/g)* | (weight %) |
| Solₛₜₐᵣₜ | None | | | No | | 3.4 | — | — |
| | | | | extraction | | | | |
| 1 | FA | 49/51 | 20 | 2.0 | 20 | 119.3 | 17.6 | 61.2 |
| 2 | FA | 20/80 | 20 | 2.0 | 20 | 66.5 | 9.7 | 37.6 |
| 3 | AA | 61/39 | 22 | 2.1 | 20 | 13.7 | 45.7 | 85.3 |
| 4 | AA | 66/34 | 305 | 30.0 | 60 | 6.9 | nd | nd |
| 5 | PA | 53/47 | 26 | 2.1 | 20 | 6.1 | 57.3 | 93.0 |
| 6 | PA | 36/64 | 38 | 2.1 | 20 | 12.7 | 27.8 | 76.6 |
| 7 | PA | 20/80 | 101 | 3.2 | 20 | 20.0 | 8.8 | 73.9 |
| 8 | FA/AA | 68/32 | 20 | 2.0 | 20 | 52.7 | 37.6 | 75.7 |

Solₛₜₐᵣₜ = Starting solution before extraction (reaction mixture before extraction)
8COA = 8-Cyanooctanoic acid
ΣNitrileₛₐₜ = Sum of the saturated nitriles = myristic nitrile + palmitic nitrile + stearic nitrile + arachidic nitrile
*Total amount of extract by the acid/water phase measured after evaporation of carboxylic acid/water, the “dry extract” being the solids in the sense that there is no solvent.

Table 4 shows that 8-cyanooctanoic acid (8COA) is extracted preferentially relative to the saturated nitriles. The 8COA/nitrileₛₐₜ mole ratio increases in the aqueous phase extracted with said water-acid mixture. The selectivity decreases with the chain length of the carboxylic acid. The selectivity decreases when the extraction temperature increases.

The amount extracted (globally) increases with the chain length of the carboxylic acid. The addition of water to the carboxylic acid reduces the amount extracted, in other words increasing the water/acid ratio decreases this extraction amount or yield.

### Example 2

### Extraction of a Diacid (Azelaic Acid: AZ) from a Reaction Mixture R2 Via the Oxidative Cleavage of Oleic Acid

The reaction for the oxidative cleavage of oleic acid is performed in a similar manner to that for the oxidative cleavage of oleonitrile (replacement with oleic acid). The oleic acid used is Oleon at a purity of 75%. The reaction takes place at 70° C. without a stream of air and with 144% pure H₂O₂ relative to the pure oleic acid.

A sample of reaction mixture R2 derived from the oxidative cleavage of oleic acid was subjected to extraction with a solvent mixture: acetic acid (AA)/water corresponding to AA/water=58/42 by weight.

A weight of 2.1 g of solution (R2) derived from said oxidative cleavage was placed in contact with a weight of 24.9 g of said AA/water solvent mixture. Thus, 36.9% of the azelaic acid present at the start was extracted by this solvent mixture. The composition of the extract is given in table 5 below. The weight ratio of azelaic acid to fatty acid (14:0, 16:0, 18:0) before extraction is 2.3, and it increases to 28.8 after the extraction.

Table 5 below gives the composition of the oleic acid cleavage solution and its extract with 58/42 acetic acid (AA)/water (weight ratio).

TABLE 5
composition before extraction in reaction mixture
R2 and after extraction in the dry extract

| | | Dry extract |
|---|---|---|
| | Before extraction | (100% material without |
| Component | (weight %) | solvent) (weight %) |
| Hexanoic acid (A6) | 0.7 | 0.7 |
| Nonanal | 1.1 | 0.2 |
| A7 | 2.3 | 2.3 |
| A8 | 1.2 | 1.3 |
| A9 | 17.5 | 17.6 |
| A10 | 0.3 | 0.2 |
| Suberic acid | 0.3 | 0.7 |
| Azelaic acid | 16.0 | 31.3 |
| C14:0 | 1.9 | 0.5 |
| Sebacic acid | 0.4 | 0.7 |
| Undecanoic acid (UA) | 2.1 | 3.7 |
| C16:0 | 3.8 | 0.6 |
| Dodecanoic acid | 0.3 | 0.5 |
| C18:0 | 1.2 | 0.0 |

1. A process for the selective separation of a reaction product from the reaction mixture and relative to reaction byproducts and unreacted or unreactive residual starting materials, characterized in that:
a) said reaction product is at least one ω-functionalized acid, selected from diacids, acid esters and acid nitriles and comprises from 6 to 15 carbon atoms,
b) said product is derived from a reaction for the oxidative cleavage of at least one unsaturated fatty acid, including a hydroxylated fatty acid or ester derivative thereof, including a monoester or multiester of a polyol, including glycerol or the nitrile derivative thereof,
c) said process comprises at least one step and, of selective extraction of said ω-functional acid product with a selective extraction solvent, which is a composition comprising a mixture of water and of at least one carboxylic acid containing 1 to 4 carbon atoms or mixtures thereof, preferably 1 to 3 carbon atoms or mixtures thereof, and more preferentially in a water/acid ratio such that, at the extraction temperature, the mixture of said reaction mixture with said extraction solvent is biphasic (two-phases), said reaction product, the ω-functional acid, being selectively concentrated in the aqueous extraction phase and said reaction byproducts and unreacted or unreactive residual starting materials being concentrated in the organic phase at each extraction step,
d) said process comprises the treatment of said aqueous extraction phase, optionally cumulated over several extractions with removal by evaporation of said extraction solvent, water/acid and recovery of said product with the dry extract thus obtained, optionally, said product possibly being recovered by liquid-liquid back-extraction of said aqueous phase with a solvent for said product which is immiscible with said aqueous phase.

2. The process as claimed in claim 1, wherein, in said extraction solvent for said extraction step c), said water/carboxylic acid weight ratio ranges as a function also of the extraction temperature, from 32/68 to 95/5.

3. The process as claimed in claim 1, wherein said extraction takes place at a temperature ranging from the melting point to the boiling point of said water/carboxylic acid mixture.

4. The process as claimed in claim 1, wherein said extraction takes place at room temperature (20±5° C.).

5. The process as claimed in claim 1, wherein the temperature of said extraction step c) and said water/acid ratio are chosen such that, during this extraction step, the mixture of the water/acid composition and of the reaction mixture (organic reaction phase) comprising said product to be separated out is two-phase with an aqueous phase (water-acid) selectively and predominantly containing said product to be separated out and a nonaqueous phase containing the rest of the organic phase.

6. The process as claimed in claim 1, wherein said extraction step c) comprises several successive washes-extractions of said nonaqueous (organic) phase with said water/acid composition with recovery and mixing of all the aqueous phases thus obtained before said treatment d) to recover said ω-functional acid product, with a weight ratio of the water/acid composition to the weight of the organic phase to be extracted ranging from 0.5 to 100/1 and with this ratio being reduced with the number of successive extractions.

7. The process as claimed in claim 1, wherein it forms an integral part of the process for manufacturing said ω-functionalized acid product.

8. The process as claimed in claim 1, wherein in that the purity of said unsaturated fatty acid or the ester or nitrile derivative thereof does not exceed 95%, preferably does not exceed 90% by weight.

9. A process for manufacturing an ω-functionalized acid, which is an α,ω-diacid, an ω-acid ester or an ω-acid nitrile or a derivative of these products wherein it comprises the use of a selective separation process, as defined in claim 6, which manufacturing process comprises a step, prior to this use, of a reaction for the oxidative cleavage of an unsaturated starting material, from which reaction are derived said ω-functionalized product and said unreacted or unreactive residual starting materials and byproducts, said starting material subjected to said oxidative cleavage being chosen from at least one unsaturated fatty acid, containing at least 10 carbon atoms and/or an ester derived from said acid and/or a nitrile derived from said unsaturated fatty acid of formula (I) below:
[R1-CH═CH—[(CH₂)_{q}—CH═CH]ₘ—(CH₂)ₜ]ₚ—X
in which
p: is an integer equal to 1, 2 or 3 and
for p=1, X is chosen from: —CO₂H (fatty monoacid) or —CN (nitrile) or —CO₂R′(monoester) with R′ being a C₁ to C₁₁ alkyl,
for p=2, X is a diester radical ═(CO₂)₂—Y, with Y being a diol residue or glycerol residue bearing an OH function,
for p=3, X is a triester ≡(CO₂)₃—Z, with Z being a glycerol residue (trivalent radical without OH) (in the case of a fatty acid oil),
R1: is an H or an alkyl radical of 1 to 11 carbon atoms, where appropriate comprising an OH function,
q: is equal to 0 or 1,
m: is equal to 0, 1 or 2,
r: is an integer ranging from 4 to 15,
with the C═C unsaturations of said formula possibly being of cis or trans conformation.

10. The process as claimed in claim 9, wherein said unsaturated starting material is chosen from a fatty acid or a fatty acid ester, preferably from: oleic acid, oleic oil or an oleic acid monoester, more preferentially oleic acid.

11. The process as claimed in claim 9, wherein said unsaturated starting material is a nitrile from oleonitrile or octadec-11-enoic nitrile and octadec-12-enoic nitrile or mixtures thereof synthesized from ricinoleic acid after hydrogenation, followed by dehydration.

12. The process as claimed in claim 11, wherein said nitrile is the product of ammoniation (reaction with ammonia) of the corresponding fatty acid or of an ester of said fatty acid and in particular of the oil thereof.

13. The process as claimed in claim 9, wherein said oxidative cleavage reaction step is performed using as oxidative cleavage agent hydrogen peroxide, oxygen and/or ozone.

14. The process as claimed in claim 11, wherein said unsaturated starting material is oleonitrile and said reaction product is 8-cyanooctanoic acid.

15. The process as claimed in claim 11, wherein said starting material is gondoic acid (eicos-11-enoic acid) nitrile or vaccenic acid (octadec-11-enoic acid) nitrile and in that the reaction product obtained is 10-cyanodecanoic acid.

16. The process as claimed in claim 1, wherein said reaction byproducts and/or heavier unreacted or unreactive residual starting materials comprise:
saturated fatty acids, esters or nitriles of the same rank as said unsaturated starting material,
the residual unsaturated starting material of the diol or epoxy derivatives of said unsaturated starting material, formed by oxidative modification other than oxidative cleavage of the ethylenic unsaturation of said unsaturated starting material, higher saturated and/or unsaturated fatty acids.

17. A process for manufacturing an ω-functionalized acid, which is an α,ω-diacid, an ω-acid ester or an ω-acid nitrile or a derivative of these products, wherein t comprises the use of a selective separation process, which manufacturing process comprises a step, prior to this use, of a reaction for the oxidative cleavage of an unsaturated starting material, from which reaction are derived said ω-functionalized product and unreacted or unreactive residual starting materials and byproducts, said starting material subjected to said oxidative cleavage being chosen from at least one unsaturated fatty acid, containing at least 10 carbon atoms and/or an ester derived from said acid and/or a nitrile derived from said unsaturated fatty acid of formula (I) below:
[R1-CH═CH—[(CH₂)_{q}—CH═CH]ₘ—(CH₂)ᵣ]ₚ—X
in which
p: is an integer equal to 1, 2 or 3 and
for p=1, X is chosen from: —CO₂H (fatty monoacid) or —CN (nitrile) or —CO₂R′ (monoester) with R′ being a C₁ to C₁₁ alkyl,
for p=2, X is a diester radical ═(CO₂)₂—Y, with Y being a diol residue or glycerol residue bearing an OH function,
for p=3, X is a triester ≡(CO₂)₃—Z, with Z being a glycerol residue (trivalent radical without OH) (in the case of a fatty acid oil),
R1; is an H or an alkyl radical of 1 to 11 carbon atoms, where appropriate comprising an OH function,
q: is equal to 0 or 1,
m: is equal to 0, 1 or 2,
r: is an integer ranging from 4 to 15,
with the C═C unsaturations of said formula possibly being of cis or trans conformation the manufacturing process concerns the preparation of an ω-amino acid comprising from 6 to 15 carbon atoms, wherein it is obtained from its ω-cyano acid precursor, said precursor being obtained via a process as defined and claimed in claim 1 and said manufacturing process comprising an additional step of hydrogenation of said ω-cyano acid precursor to obtain said amino acid.

18. The process as claimed in claim 17, wherein said ω-cyano acid is 8-cyanooctanoic acid and said ω-amino acid derivative is 9-aminononanoic acid or in that said cyano acid is 10-cyanodecanoic acid and in that said amino acid derivative is 11-aminoundecanoic acid.

19-22. (canceled)

23. The process as claimed in claim 1, wherein the said carboxylic acid has 1 to 3 carbon atoms and is selected from formic or acetic acid or their mixture.

24. The process as claimed in claim 23, wherein the said carboxylic acid is a mixture of formic and acetic acid.

25. The process as claimed in claim 2, wherein the said ratio of water/carboxylic acid is from 38/62 to 85/15.

26. The process as claimed in claim 13, wherein said oxidative agent is hydrogen peroxide.
